(19) United States
(12) Patent Application Publication
(10) Pub. No.: US 2009/0233280 A1
(43) Pub. Date: Sep. 17, 2009

(54) METHOD OF ACQUIRING INFORMATION REGARDING BASE SEQUENCE AND INFORMATION READING DEVICE FOR THE SAME

(75) Inventors: Tsuyoshi Nomoto, Tokyo (JP); Wataru Kubo, Inagi-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/093,138

(22) PCT Filed: Dec. 26, 2006
(86) PCT No.: PCT/JP2006/326349

§ 371 Date: May 8, 2008

(30) Foreign Application Priority Data
Dec. 28, 2005 (JP) 2005-380326

(51) Int. Cl. C12Q 1/68

(52) U.S. Cl. 435/6

(57) ABSTRACT

Information regarding a base sequence of a target nucleic acid is acquired by preparing a sample comprising the target nucleic acid hybridized with a primer or a sample comprising the target nucleic acid containing a promoter sequence, a polymerase, a nucleotide derivative having an electrochemically convertible moiety; allowing the three components to coexist in a solvent; and detecting whether the nucleotide derivative is introduced into the primer or a transcription product of the target nucleic acid or not using an electrochemical reaction.

### TECHNICAL FIELD

The present invention relates to an information acquiring method for acquiring information regarding bases included in a target nucleic acid.

### BACKGROUND ART

The dideoxy method is known to be one of methods of analyzing base sequences of nucleic acids (Proceedings of National Academy of Sciences, USA, 74:5463-5467, 1977). As a method of determining nucleic acid base sequences using the dideoxy method, the method described in Japanese Patent Application Laid-Open No. H05-168500 is known.

### DISCLOSURE OF THE INVENTION

The method of analyzing base sequences using the dideoxy method which is described in Japanese Patent Application Laid-Open No. H05-168500 includes a step of separating elongated DNA strands by electrophoresis, so it requires a long period of time to obtain an analysis result.

Therefore, the inventors of the present invention have made extensive studies on a method which enables acquiring information regarding a base sequence in a shorter period of time than in a case of using electrophoresis, and as a result, have come with the present invention.

That is, it is an object of the present invention to provide an information acquiring method which enables acquisition of information regarding a base sequence in a shorter period of time than in a case of using electrophoresis, and a device for the same.

A method of acquiring information regarding a base sequence according to the present invention includes: preparing multiple kinds of nucleotide derivatives each having a removable moiety which is electrochemically removable from the nucleotide derivatives, a double-stranded sample made of a target nucleic acid and a primer- and a polymerase; elongating the primer by one base with one kind of the nucleotide derivatives by allowing the sample, the polymerase, and the nucleotide derivatives to coexist in a solvent; applying a voltage to the sample; and detecting an electric signal due to removal of the removable moiety from the nucleotide derivative which has been incorporated in the primer.

In addition, a method of acquiring information regarding a base sequence according to another aspect of the present invention includes: preparing a sample comprising a target nucleic acid hybridized with a primer or a sample comprising a target nucleic acid containing a promoter sequence, a polymerase, and a nucleotide derivative having an electrochemically convertible moiety; allowing the sample, the polymerase, and the nucleotide derivative to coexist in a solvent; and detecting whether the nucleotide derivative is introduced into the primer or a transcription product of the target nucleic acid or not by electrochemically converting the electrochemically convertible moiety.

In addition, a method of acquiring information regarding a base sequence according to still another aspect of the present invention includes: preparing a sample comprising a target nucleic acid hybridized with a primer or a sample comprising a target nucleic acid containing a promoter sequence, a polymerase, and multiple kinds of nucleotide derivatives each of which has an electrochemically convertible moiety and which are electrochemically distinguishable from one another; allowing the sample, the polymerase, and the multiple kinds of the nucleotide derivatives to coexist in a solvent; and identifying, among the multiple kinds of the nucleotide derivatives, a nucleotide derivative which has been introduced into the primer or a transcription product of the target nucleic acid by electrochemically converting the electrochemically convertible moiety thereof.

In addition, a method of acquiring information regarding a base sequence according to still another aspect of the present invention is characterized by including: allowing a polymerase, a sample comprising a target nucleic acid hybridized with a primer or a sample containing a promoter sequence for the polymerase, and a nucleotide derivative having an electrochemically convertible moiety to coexist in a solvent; and detecting an electric signal through an electroconductive member which is electrically connected to the solvent.

Here, the electric signal refers to a signal generated by electrochemical conversion of the above-mentioned moiety of the nucleotide derivative which has been introduced into the primer or a transcription product of the target nucleic acid.

In addition, the above-mentioned moiety of the nucleotide derivative is characterized by being a moiety which undergoes a reaction of removal from the nucleotide derivative, substitution in the nucleotide derivative, or addition to the nucleotide derivative owing to the electrochemical conversion.

In addition, a device for acquiring information regarding a base sequence according to the present invention includes: a voltage applying section for applying a voltage to a sample which contains a nucleotide derivative having an electrochemically convertible moiety; and an electric signal acquiring section for acquiring an electric signal due to electrochemical conversion of the moiety of the nucleotide derivative.

Note that it is also preferable that the device for acquiring information regarding a base sequence further include an identifying section for identifying the nucleotide derivative using the signal from the electric signal acquiring section.

In addition, an article according to the present invention includes: an electroconductive member; and a polymerase immobilized onto the electroconductive member.

In addition, the nucleotide derivative according to the present invention is characterized by having an electrochemically convertible moiety, and undergoing a reaction of removal, substitution, or addition owing to the electrochemical conversion of the electrochemically convertible moiety.

In addition, a base sequence information acquiring method for acquiring information regarding a base at an information acquiring position in a target nucleic acid according to the present invention includes the steps of:

(1) preparing: the target nucleic acid; a primer which recognizes at least a part of a 3′-side region including a base adjacent in a 3′-direction to the base at the information acquiring position in the target nucleic acid, and which hybridizes with the 3′-side region; a polymerase; and a nucleotide derivative having an electrochemically convertible moiety;

(2) hybridizing the 3′-side region including the base adjacent in a 3′-direction to the base at the information acquiring position in the target nucleic acid, with the primer;

(3) allowing the target nucleic acid having the hybridized region to coexist with the nucleotide derivative under a presence of the polymerase; and

(4) acquiring information regarding the base at the information acquiring position by detecting occurrence or absence of incorporation of the nucleotide derivative into at a 3′-terminal position of a strand including the primer, which position corresponds to the information acquiring position, based on electrochemical conversion of the electrochemically convertible moiety held by the nucleotide derivative.

In addition, a base sequence analyzing method for analyzing a base sequence of a subject region to be analyzed in a target nucleic acid of the present invention includes the steps of:

(1) preparing: the target nucleic acid; a primer which recognizes a 3′-side region including a base adjacent in a 3′-direction to the subject region to be analyzed in the target nucleic acid, and which hybridizes with the 3′-side region; a DNA polymerase; and a set of nucleotide derivatives including a 2′-deoxyadenosine 5′-triphosphate derivative, a 2′-deoxycytidine 5′-triphosphate derivative, a 2′-deoxyguanosine 5′-triphosphate derivative, and a 2′-deoxythymidine 5′-triphosphate derivative each of which has an electrochemically convertible moiety and which are electrochemically distinguishable from one another;

(2) hybridizing the target nucleic acid with the primer;

(3) elongating the primer by one base by allowing the target nucleic acid hybridized with the primer, the set of nucleotide derivatives, and the DNA polymerase to coexist so that a nucleotide derivative selected from the set of nucleotide derivatives is incorporated into a 3′-terminal of the primer hybridized with the target nucleic acid;

(4) identifying a base in the subject region to be analyzed which is present at a position corresponding to the nucleotide derivative which has been incorporated into the 3′-terminal of the elongated primer, by identifying the incorporated nucleotide derivative based on electrochemical conversion of the electrochemically convertible moiety in the incorporated nucleotide derivative; and

(5) repeating the steps (3) and (4) depending on the number of bases when the number of bases in the subject region to be analyzed is 2 or more, in which an additional elongation reaction of the elongated primer by the DNA polymerase is inhibited after the primer is elongated by one base in the step (3) owing to a presence of the electrochemically convertible moiety, and an additional elongation reaction of the elongated primer by the DNA polymerase is allowed to proceed after the electrochemical conversion of the electrochemically convertible moiety in the step (4) owing to a reaction of removal from, substitution in, or addition to the incorporated nucleotide derivative.

In addition, a base sequence analyzing method for analyzing a base sequence of a subject region to be analyzed in a target nucleic acid according to the present invention includes the steps of:

(1) preparing: an RNA polymerase; a target nucleic acid containing a promoter sequence for the RNA polymerase; and a set of nucleotide derivatives including an adenosine 5′-triphosphate derivative, a cytidine 5′-triphosphate derivative, a guanosine 5′-triphosphate derivative, and a uridine 5′-triphosphate derivative each of which has an electrochemically convertible moiety and which are electrochemically distinguishable from one another;

(2) transcribing by one base the target nucleic acid which is located downstream of the promoter sequence by allowing the target nucleic acid, the set of nucleotide derivatives, and the RNA polymerase to coexist so that a nucleotide derivative selected from the set of nucleotide derivatives is incorporated into the target nucleic acid;

(3) identifying a base in the target nucleic acid, which is present at a position corresponding to the nucleotide derivative which has been incorporated into a 3′-terminal of the transcription product, by identifying the incorporated nucleotide derivative based on electrochemical conversion of the electrochemically convertible moiety in the incorporated nucleotide derivative; and

(4) repeating the steps (2) and (3) depending on the number of bases when the number of bases in the subject region to be analyzed is 2 or more, in which an additional transcription reaction by the RNA polymerase is inhibited after the target nucleic acid is transcribed by one base in the step (2) owing to a presence of the electrochemically convertible moiety, and an additional transcription reaction by the RNA polymerase is allowed to proceed after the electrochemical conversion of the electrochemically convertible moiety in the step (3) owing to a reaction of removal from, substitution in, or addition to the incorporated nucleotide derivative.

A device for acquiring information regarding a base sequence of a subject region to be analyzed in a target nucleic acid according to the present invention includes at least: a reaction section for allowing a nucleotide derivative having an electrochemically convertible moiety to react with a sample in which a primer is hybridized with a 3′-side region containing a base adjacent in a 3′-direction to the subject region to be analyzed in the target nucleic acid, or a sample of the target nucleic acid containing a promoter sequence for an RNA polymerase; an electrode system including a polymerase immobilized electrode, a counter electrode, and a reference electrode which are arranged in the reaction section; a voltage controlling section for a voltage to be applied to the electrode system; and a computer which processes, as data, changes with time of the voltage applied to the electrode system and of an electric current which flows the electrode system.

According to the present invention as described above, information regarding a base sequence can be acquired without a step of separating an elongated DNA strand by electrophoresis, so the time period required for the electrophoresis can be omitted.

Note that, however, the present invention is not intended to exclude any methods each of which involve acquiring information regarding a base sequence using electrophoresis and devices for the methods. For example, the present invention is not intended to exclude a combination of a procedure of electrochemically converting a moiety of a nucleotide derivative, which is a characteristic constituent of the present invention, and a procedure using electrophoresis.

In addition, the term “electrochemical conversion” as used in the present invention means removal from, substitution in, or addition to a nucleotide derivative of its moiety caused by giving or receiving of electrons via an electroconductive substrate, with cleavage and reconstitution of a chemical bond which are elicited by the giving and receiving of electrons.

Further features of the present invention will become apparent from the following description of exemplary embodiments.

### BRIEF DESCRIPTION OF THE DRAWINGS

FIGS. 1A, 1B, 1C and 1D are schematic diagrams showing respective steps of a method of analyzing a base sequence of a nucleic acid of the present invention.

FIG. 2 is a schematic diagram showing an exemplary constitution of a DNA base sequence analyzer for performing the method of analyzing a base sequence of a nucleic acid of the present invention.

FIGS. 3A and 3B are schematic diagrams showing data acquired in Example 2 of the present invention.

FIGS. 4A and 4B are views each showing the position in a target nucleic acid, which is recognized by a primer.

FIG. 5 is an exemplary flow chart showing a flow of a program which is executed in the DNA base sequence analyzer of the present invention.

### BEST MODE FOR CARRYING OUT THE INVENTION

Hereinafter, the present invention will be described in detail.

### A) First Embodiment

The present invention can be applied, for example, to a case of determining a base sequence in a certain portion.

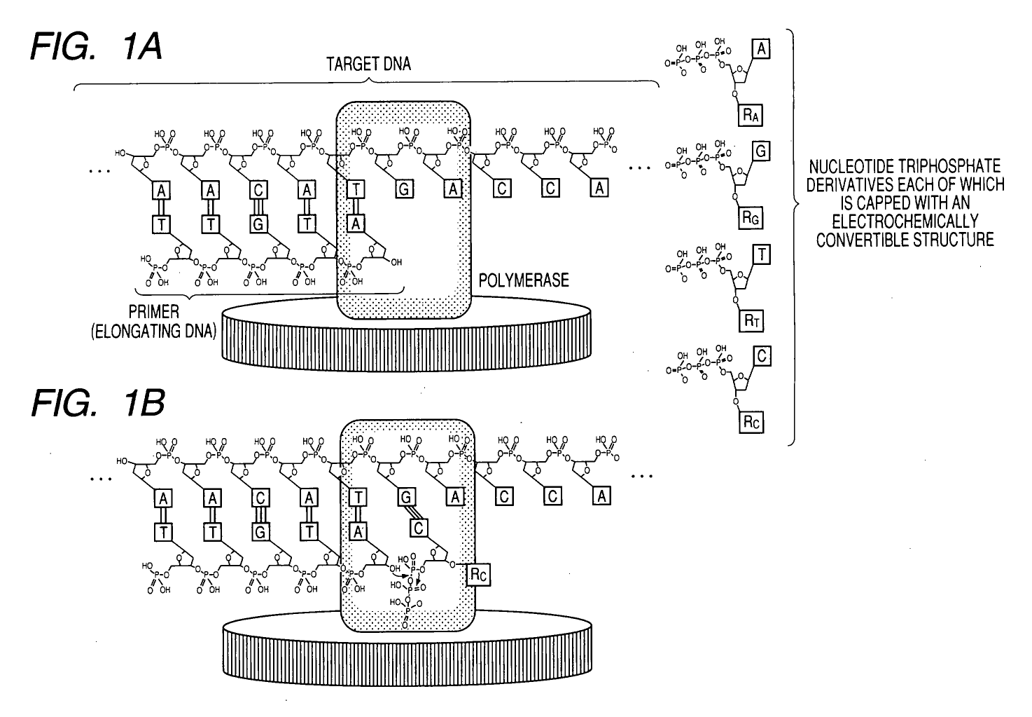

First, a double-stranded sample made of a target nucleic acid and a primer (sample comprising a target nucleic acid hybridized with a primer), a polymerase, and nucleotide derivatives each of which has an electrochemically convertible moiety are prepared (FIG. 1A).

The nucleotide derivatives are, for example, nucleoside 5′-triphosphate derivatives, and at least include the following:

adenosine 5′-triphosphate derivative;

cytidine 5′-triphosphate derivative;

guanosine 5′-triphosphate derivative;

uridine 5′-triphosphate derivative;

2′-deoxyadenosine 5′-triphosphate derivative;

2′-deoxycytidine 5′-triphosphate derivative;

2′-deoxyguanosine 5′-triphosphate derivative; and

2′-deoxytimidine 5′-triphosphate derivative.

Hereinafter, even in the inventions following the section B), the materials exemplified above as the nucleotide derivatives are appropriately used.

Note that, although FIG. 1A shows multiple kinds of nucleotide derivatives, multiple kinds or a single kind thereof may be used depending on requirements.

Then, the sample, the polymerase, and the nucleotide derivatives are allowed to coexist in a solvent (FIG. 1A). In this regard, there may be cases where the nucleotide derivative is introduced into the 3′-terminal of the primer (FIGS. 1B and 1C) and where the nucleotide derivative is not introduced thereinto.

After that, detection is conducted using an electrochemical reaction to determine whether the nucleotide derivatives are introduced into the primer or not. Specific procedures of the detection will be described below.

For example, if a nucleotide derivative is introduced into a certain position in a sample when only the nucleotide derivative is allowed to coexist with a sample, a base corresponding to the nucleotide derivative will be found to be present on the certain position in the sample. If no nucleotide derivative is found to be introduced into the certain position in the sample from the determination, the base corresponding to the nucleotide derivative will be found not to be present at least on the certain position in the sample.

In this manner, information regarding a base sequence of a target nucleic acid is acquired.

Here, the information includes, of course, what kind of base, that is, adenine (A), cytosine (C), guanine (G), thymine (T), and the like, is present at the position intended to be identified, but also includes that at least a kind of base is not present at the position (for example, information that the base is not A). The same holds true for the embodiments described below.

Note that the term “solvent” refers to an aqueous solution, a gel-like substance, or the like.

In addition, the term “conversion” of the electrochemically convertible moiety of a nucleotide derivative means cleavage and reconstitution of a chemical bond, which is generated by giving and receiving of electrons via an electroconductive substrate.

The term “conversion” includes reactions of removal, substitution, and addition of the above-mentioned moiety and a nucleotide derivative of high-order group containing the above-mentioned moiety.

In other words, the electrochemically convertible moiety in the nucleotide derivative refers to a moiety which is removed from the nucleotide derivative, substitutes for a moiety of the nucleotide derivative, or is added to become a moiety of the nucleotide derivative owing to the electrochemical reaction.

Meanwhile, though metal complexes may be given as examples of an electrochemically active functional group (that is, functional group that gives and receives electrons to and from an electroconductive substrate), substances, like the metal complexes, which only undergo a change in oxidation number due to oxidation or reduction of a central metal thereof without causing any cleavage and reconstitution of a chemical bond thereof are not regarded as the electrochemically convertible moiety in the present invention. The same holds true for the embodiments described below.

### B) Second Embodiment

A description will be made of a method of reading a base sequence of a target nucleic acid.

First, a double-stranded sample made of a target nucleic acid and a primer (sample comprising a target nucleic acid hybridized with a primer), a polymerase, and multiple kinds of nucleotide derivatives each of which has an electrochemically convertible moiety and which are electrochemically distinguishable from one another are prepared.

The phrase “be electrochemically distinguishable” means that the number of electrons, the voltage to be applied, and the like which are required for the electrochemical conversion of the nucleotide derivatives differ among the nucleotide derivatives so that the nucleotide derivatives can be distinguished by usual electrochemical determination means.

The sample, the polymerase, and the multiple kinds of the nucleotide derivatives are allowed to coexist in a solvent so that a nucleotide derivative having a base complementary to a base of the target nucleic acid is polymerized at the 3′-terminal of the primer. An additional elongation reaction of the primer by the polymerase should be inhibited at this time by the presence of the electrochemically convertible moiety which has been introduced into the nucleotide derivative. This enables synthetic reactions as a whole to proceed in synchronization because, even if multiple molecules of the target nucleic acid were present, the polymerization reaction is terminated as the primer is elongated by one base.

Then, among the multiple kinds of the nucleotide derivatives, the nucleotide derivative which has been introduced into the 3′-terminal of the primer is identified by electrochemically converting the electrochemically convertible moiety thereof.

At this time, the electrochemically convertible moiety which has been introduced into the nucleotide derivative should be made to enable the elongation reaction by the polymerase to proceed again owing to a reaction of removal, substitution, or addition caused by the electrochemical conversion thereof.

After the identification, the elongation reaction by the polymerase spontaneously starts again. Thus, the base sequence of the target nucleic acid can be successively read by repeating the step for next identification after awaiting a period of time required for the polymerase to elongate the primer by one base has passed. In general, the elongation reaction by the polymerase terminates in 1/500 second per base, so the base sequence can be read at high speed.

### C) Third Embodiment

First, a double-stranded sample made of a target nucleic acid and a primer (sample comprising a target nucleic acid hybridized with a primer), a polymerase, and a nucleotide derivative having an electrochemically convertible moiety are allowed to coexist in a solvent.

Then, an electric signal is detected through an electroconductive member which is electrically connected to the solvent.

The term “electric signal” as used herein refers to a signal generated by electrochemical conversion of the abovementioned moiety of the nucleotide derivative which has been introduced into the primer.

### D) Fourth Embodiment

A device for acquiring information regarding a base sequence is composed of the following constitution.

The information acquiring device has a voltage applying section (for example, a first electrode comprising an electroconductive member) for applying a voltage to a sample containing a nucleotide derivative having an electrochemically convertible moiety.

Further, the information acquiring device has an electric signal acquiring section (for example, a second electrode) for acquiring an electric signal generated by electrochemical conversion of the moiety of the nucleotide derivative.

Further, the information acquiring device preferably has an identifying section for identifying the nucleotide derivative by using the signal from the electric signal acquiring section.

### E) Fifth Embodiment

An article which comprises an electroconductive member and a polymerase immobilized onto the electroconductive member can preferably be utilized in the above-mentioned embodiments A to D.

### F) Sixth Embodiment

A nucleotide derivative which has the electrochemically convertible moiety as described above and which undergoes a reaction such as removal, substitution, or addition owing to the electrochemical conversion of the electrochemically convertible moiety can preferably be utilized in the above-mentioned embodiments A to D.

Note that FIG. 1A shows a case where a partly double-stranded sample made of a DNA as a target nucleic acid and a primer (sample comprising a target nucleic acid hybridized with a primer), multiple kinds of nucleotide derivatives, and a polymerase are prepared. FIGS. 1B and 1C show elongation steps. FIG. 1D shows removal of the electrochemically convertible moiety held by the nucleotide derivative after the elongation.

In the first to sixth embodiments of the present invention, there can be used particularly an RNA polymerase and a sample as exemplified below instead of a sample comprising a target nucleic acid hybridized with a primer.

For example, a sample containing a promoter sequence for the RNA polymerase can be used. This sample and a nucleotide derivative having an electrochemically convertible moiety can be used for performing a transcription reaction, to thereby detect introduction of the nucleotide derivative into a transcription product.

Hereinafter, the present invention will be described more in detail.

Examples of the target nucleic acid to be used in the present invention include DNAs, RNAs, oligodeoxyribonucleotides, and oligoribonucleotides. The target nucleic acid may be single-stranded or double-stranded. In addition, the target nucleic acid is not necessarily purified, and biological samples containing the target nucleic acid may be used.

The primer to be used in the present invention is an oligonucleotide which hybridizes with a target nucleic acid when the target nucleic acid is a DNA or an RNA.

The length of the primer is not limited, but it is desirable that the primer be an oligonucleotide having a length of preferably about 15 mer to 60 mer.

The primer is used for making a region upstream in the 3′-direction (3′-side region) of a base sequence intended for acquiring information of the target nucleic acid into a double strand (hybridization). The primer may be one having a base sequence that recognizes the entire 3′-side region, or may be one that recognizes a part of the 3-side region. In a case where the primer that recognizes a part of the 3′-side region is used, the primer is subjected to an elongation reaction up to a position corresponding to a base which is adjacent in the 3′-direction (upstream) to the base that is the information acquiring target in the target nucleic acid, as required, and the elongated primer is then used for an incorporation reaction of the nucleotide derivative using the polymerase as described below.

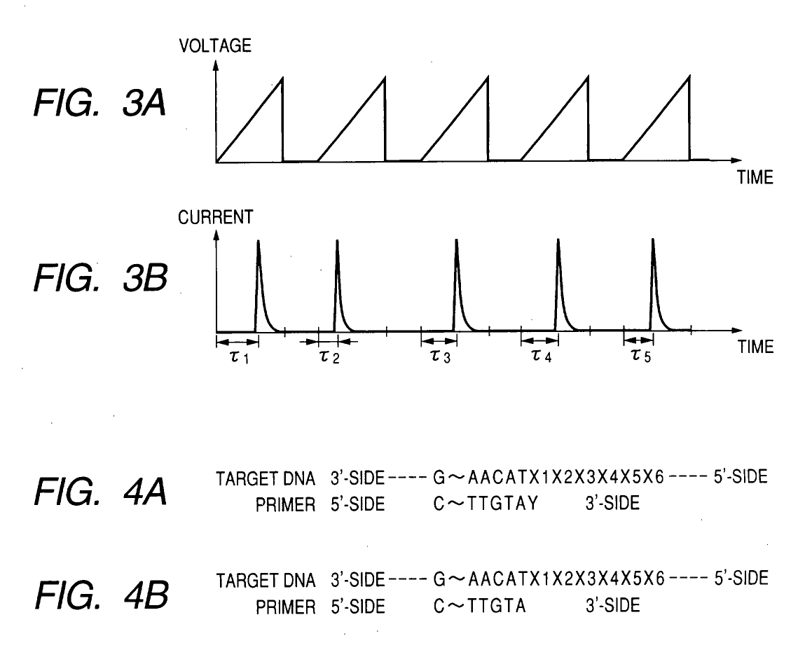

For example, as shown in FIG. 4A, when the 3′-side region in a target DNA, which is recognized by a primer (region on the 3′-side of the base X1 that is the information acquiring target) is “G˜AACAT”, a primer composed of a base sequence complementary thereto, that is, “C˜TTGTA” is bound to the 3′-side region, to thereby make the 3′-side region double-stranded.

Alternatively, as shown in FIG. 4B, a primer “C˜TTGT” which recognizes a part of the 3′-side region including a base upstream by 2 bases of the X1 is bound to the 3′-side region, and then an elongation reaction is performed to thereby add “A” to the primer and make the entire 3′-side region of the base X1 that is the information acquiring target double-stranded.

Note that, in the case of FIG. 4B, when a primer which recognizes a part of the 3′-side region to a base upstream by 3 bases of the X1, the 3′-terminal of the primer is successively subjected to an elongation reaction up to the base corresponding to the base upstream of the X1 by one base.

An example of an information acquiring method using the primer having the base sequence that recognizes the entire 3′-side region is a method including the following steps.

(1) A step of preparing: a target nucleic acid; a primer for making a 3′-side region double-stranded, which recognizes the entire 3′-side region including the base adjacent in the 3′-direction to the base at an information acquiring position in a base sequence of the target nucleotide; a polymerase; and a nucleotide derivative having a substituent for determination.
(2) A step of forming a double strand by binding the primer to the 3′-side region including the base adjacent in the 3′-direction to the base at the information acquiring position in the base sequence of the target nucleotide.
(3) A step of allowing the target nucleic acid having the double-stranded portion to coexist or react with the nucleotide derivative under the presence of the polymerase.
(4) A step of acquiring information regarding the base which is present at the information acquiring position by using the substituent for determination held by the nucleotide derivative. Specifically, the occurrence or absence of incorporation of the nucleotide derivative into the position corresponding to the base at the information acquiring position, the position being located at the 3′-terminal of a strand including the primer among the two strands made of the target nucleic acid and the primer, is detected.

An example of an information acquiring method using the primer which recognizes a part of the 3′-side region is a method including the following steps.

(1) A step of preparing: a target nucleic acid; a primer which recognizes a part of the 3′-side region including the base upstream by 2 bases in the 3′-direction of the base at an information acquiring position in a base sequence of the target nucleic acid and which enables making the 3′-side region double-stranded; a polymerase; and a nucleotide having a substituent for determination.
(2) A step of binding the primer to the target nucleic acid.
(3) A step of making the 3′-side region including the base adjacent in the 3′-direction to the base at the information acquiring position in the base sequence of the target nucleic acid by allowing the 3′-side terminal of the primer bound to the target nucleic acid to elongate.
(4) A step of allowing the target nucleic acid to which the elongated primer is bound to react with the nucleotide derivative under the presence of the polymerase.
(5) A step of acquiring information regarding the base at the information acquiring position by using the substituent for determination held by the nucleotide derivative. Specifically, the occurrence or absence of incorporation of the nucleotide derivative into the position corresponding to the base at the information acquiring position of the target nucleic acid is detected.

In the present invention, the kind of the polymerase is selected depending on the kinds of the target nucleic acid and the nucleic acid to be elongated. In a case where the nucleic acid to be elongated is a DNA, a DNA polymerase (nucleic acid-dependent DNA polymerase) is selected. In a case where the nucleic acid to be elongated is an RNA, an RNA polymerase (nucleic acid-dependent RNA polymerase) is selected.

In a case where the target nucleic acid is a DNA or an oligodeoxyribonucleotide, a DNA-dependent DNA polymerase or a DNA-dependent RNA polymerase is selected and used.

On the other hand, in a case where the target nucleic acid is an RNA or an oligoribonucleotide, an RNA-dependent DNA polymerase or an RNA-dependent RNA polymerase is selected and used.

The DNA-dependent DNA polymerase which can be used in the present invention is classified into enzyme number EC 2.7.7.7, and the origin thereof is not limited as long as the DNA-dependent DNA polymerase is an enzyme which catalyzes the following reaction:

Deoxynucleoside triphosphate+DNA(n)=diphosphate+DNA(n+1)

The DNA-dependent RNA polymerase which can be used in the present invention is classified into enzyme number EC 2.7.7.6, and the origin thereof is not limited as long as the DNA-dependent RNA polymerase is an enzyme which catalyzes the following reaction:

Nucleoside triphosphate+RNA(n)=diphosphate+RNA(n+1)

The RNA-dependent RNA polymerase which can be used in the present invention is classified into enzyme number EC 2.7.7.48, and the origin thereof is not limited as long as the RNA-dependent RNA polymerase is an enzyme which catalyzes the following reaction:

Nucleoside triphosphate+RNA(n)=diphosphate+RNA(n+1)

The RNA-dependent DNA polymerase which can be used in the present invention is classified into enzyme number EC 2.7.7.49, and the origin thereof is not limited as long as the RNA-dependent DNA polymerase is an enzyme which catalyzes the following reaction:

Deoxynucleoside triphosphate+DNA(n)=diphosphate+DNA(n+1)

In any of the above-mentioned polymerases, it is desirable that a 3′→5′ exonuclease activity be deficient.

In the present invention, the occurrence or absence of the incorporation of a nucleotide derivative into a position corresponding to the base of the information acquiring target of the target nucleic acid is determined, so the information about the base can be acquired. The occurrence or absence of the incorporation is determined by using an electrochemically convertible moiety imparted to the nucleotide derivative.

The incorporation of the nucleotide derivative having an electrochemically convertible moiety into the 3′-terminal (for example, the position “Y” as shown in FIG. 4A) of a primer or an elongated primer on the target nucleic acid is determined by incorporation of the electrochemically convertible moiety.

For example, in a case where the nucleic acid to be elongated is a DNA, at least one kind of the nucleotide derivatives as exemplified below is allowed to react with a target DNA which has been made double-stranded with a primer.

Examples of the nucleotide derivatives include a 2′-deoxyadenosine 5′-triphosphate derivative, a 2′-deoxycytidine 5′-triphosphate derivative, a 2′-deoxyguanosine 5′-triphosphate derivative, and a 2′-deoxythymidine 5′-triphosphate derivative each of which has an electrochemically convertible moiety and which are distinguishable from one another.

Here, the nucleotide derivative to be used is selected, whereby the acquisition of information regarding bases as described below can be performed.

(1) In a case where “X1” as shown in FIGS. 4A and 4B is “A”, a reaction system is added with at least one of a 2′-deoxyadenosine 5′-triphosphate derivative, a 2′-deoxycytidine 5′-triphosphate derivative, and a 2′-deoxyguanosine 5′-triphosphate derivative. As a consequence, the electrochemically convertible moiety of the added nucleotide derivative is observed not to be incorporated into the position “Y”. Thus, it can be concluded that a nucleotide complementary to the nucleotide added to the reaction system is not present at “X1”.
(2) In a case where “X1” as shown in FIGS. 4A and 4B is “A”, when a reaction system is added with at least a 2′-deoxythymidine 5′-triphosphate derivative, the electrochemically convertible moiety of the 2′-deoxythymidine 5′-triphosphate derivative is incorporated into the position “Y”. The incorporation can be determined by using the electrochemically convertible moiety held by the 2′-deoxythymidine 5′-triphosphate derivative.

Note that, in a case where the nucleic acid to be elongated is an RNA, there is used as the nucleotide derivative at least one kind of the following derivatives, that is, an adenosine 5′-triphosphate derivative, a cytidine 5′-triphosphate derivative, a guanosine 5′-triphosphate derivative, and a uridine 5′-triphosphate derivative each of which has a substituent for determination and which are distinguishable from one another.

The electrochemically convertible moiety to be imparted to a nucleotide derivative causes a structural change owing to electrochemical conversion thereof.

The structural change enables an elongation reaction by the polymerase to start again, which has been stopped.

It is more preferable that the structural change be irreversible removal of the substituent containing the electrochemically convertible moiety from the nucleotide derivative.

Hereinafter, a description will be made of the nucleotide having an electrochemically convertible moiety.

The term “electrochemically convertible moiety” in the present invention refers to, for example, an atom or an atomic group which is bound to any one of atoms constituting a nucleoside 5′-triphosphate, and which has the properties as described in the following items (1) to (3).

(1) A phosphate ester bond can be formed by enzymatic catalysis of a polymerase with a hydroxyl group at the 3′-terminal of a primer or an elongated strand in a complementary pair of a target nucleic acid and a primer, or a complementary pair of a target nucleic acid and a strand elongated from a primer.

(2) Formation of an additional phosphate ester bond with other nucleoside 5′-triphosphate derivatives is inhibited after a nucleotide derivative has been bound to a hydroxyl group at the 3′-terminal of the primer or the elongated strand via the phosphate ester bond as a result of the above item (1). In other words, the nucleotide derivative to be used in the present invention functions as a capping.

(3) A moiety which can be electrochemically reduced or oxidized is contained.

Note that the nucleotide derivative preferably has a property as described in the following item (4) in addition to the above items (1) to (3).

(4) As a result of the above item (3), the nucleotide derivative undergoes a reaction of removal, substitution, or addition owing to the electrochemical reduction or oxidation thereof, and an additional phosphate ester bond can be formed by the enzymatic catalysis of the polymerase.

The capping by the electrochemically convertible moiety is classified into a removable group which is electrochemically removable and a substituent which is electrochemically substitutable depending on the difference in electrochemical properties thereof. Either of the classified groups can be used in the present invention as long as they satisfy the properties as described in the above items (1) to (3) (preferably, including the above item (4)).

The removable group which is electrochemically removable is an atom or an atomic group which undergoes removal by two-electron reduction, such as R2 shown in the formula 1 or R6 shown in the formula 3. In addition, the removable group which is electrochemically removable is an atom or an atomic group which undergoes removal by two-electron oxidation, such as R4 shown in the formula 2 or R8 shown in the formula 4.

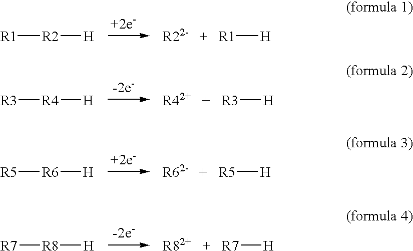

Provided that R1, R3, R5, and R7 each represent a nucleotide, and R2, R4, R6, and R8 each represent the removable group which is electrochemically removable. Specific examples of the removable group include typical metallic compounds, boric compounds, and transition organometallic complexes.

The substituent which is electrochemically substitutable is an atom or an atomic group which undergoes removal as a radical or an anion by one-electron reduction, such as R10 shown in the formulae 5 and 6. Alternatively, the substituent which is electrochemically substitutable is an atom or an atomic group which undergoes removal as a radical or an anion by one-electron oxidation, such as R12 shown in the formulae 7 and 8.

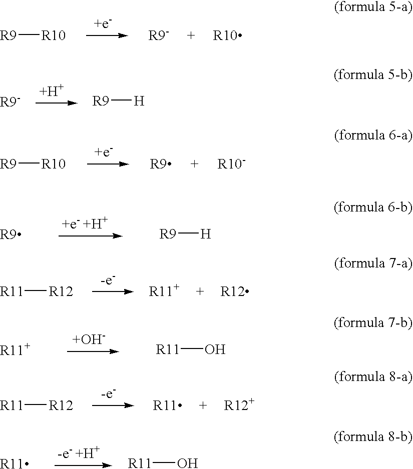

Provided that, R9 and R11 each represent a nucleotide, and R10 and R12 each represent a substituent which is electrochemically substitutable. Specific examples of the substituent include groups of halogen, alkylthio, sulfinyl, hydroxy, acyloxy, amino, peroxide, and sulfonium. In addition, examples of the substituent also include groups of organometallic complexes, nitroxy, 2,2,6,6-tetramethyl-1-piperidinyloxyl (TEMPO), hydroquinolyl, methoquinolyl, phenothiazyl, and the like.

The nucleotide derivative is a nucleoside 5′-triphosphate which is modified by the removable group which is electrochemically removable or the substituent which is electrochemically substitutable.

Specifically, the nucleotide derivative is selected depending on the kind of the polymerase to be used in the present invention. In a case where the polymerase is a DNA-dependent DNA polymerase or an RNA-dependent DNA polymerase, at least one kind of the following 4 derivatives is used:

a 2′-deoxyadenosine 5′-triphosphate derivative (dATP derivative);

a 2′-deoxycytidine 5′-triphosphate derivative (dCTP derivative);

a 2′-deoxyguanosine 51-triphosphate derivative (dGTP derivative); and

a 2′-deoxythymidine 5′-triphosphate derivative (dTTP derivative).

On the other hand, in a case where the polymerase to be used in the present invention is a DNA-dependent RNA polymerase or an RNA-dependent RNA polymerase, at least one kind of the following 4 derivatives is used:

an adenosine 5′-triphosphate derivative (ATP derivative);

a cytidine 5′-triphosphate derivative (CTP derivative);

a guanosine 5′-triphosphate derivative (GTP derivative); and

a uridine 51-triphosphate derivative (UTP derivative).

The atom to which the removable group which is electrochemically removable is added in a nucleotide derivative, that is, the atom constituting R1 in the formula 1 and R3 in the formula 3, is not particularly limited as long as the properties regarding the capping as described in the above item (1) to (4) are satisfied.

Examples of the atom for, for example, 2′-deoxyadenosine 5′-triphosphate (dATP), 2′-deoxycytidine 51-triphosphate (dCTP), 2′-deoxyguanosine 5′-triphosphate (dGTP), and 2′-deoxythymidine 5′-triphosphate (dTTP) include carbon atoms at the 1′-, 2′-, and 4′-positions and an oxygen atom of a hydroxyl group at the 3′-position of deoxyribose thereof.

In addition, examples of the atom for, for example, adenosine 5′-triphosphate (ATP), cytidine 5′-triphosphate (CTP), guanosine 5′-triphosphate (GTP), and uridine 5′-triphosphate (UTP) include oxygen atoms of hydroxyl groups at the 2′- and 3′-positions of ribose thereof.

In addition, the atom constituting R5 in the formula 3 or R7 in the formula 4 is not limited as long as all of the properties regarding the capping as described in the above items (1) to (4) are satisfied. Examples of the atom for, for example, dATP, dCTP, dGTP, and dTTP include a carbon atom at the 3′-position of deoxyribose thereof. Examples of the atom for, for example, ASP, CTP, GTP, and UTP include carbon atoms at the 2′- and 3′-positions of ribose thereof.

The atom to which the substituent which is electrochemically substitutable is added in a nucleotide derivative, that is, the atom constituting R9 in the formula 5 and R9 in the formula 6, is not limited as long as all of the properties regarding the capping as described in the above item (1) to (4) are satisfied. Examples of the atom for, for example, dATP, dCTP, dGTP, and dTTP include carbon atoms at the 1′-, 2′-, and 4′-positions and an oxygen atom of a hydroxyl group at the 3′-position of deoxyribose thereof. In addition, examples of the atom for, for example, ATP, CTP, GTP, and UTP include oxygen atoms at the 2′- and 3′-positions of ribose thereof.

In addition, the atom constituting R11 in the formula 11 or R11 in the formula 8 is not limited as long as all of the properties regarding the capping as described in the above item (1) to (4) are satisfied. Examples of the atom for, for example, dATP, dCTP, dGTP, and dTTP include a carbon atom at the 3′-position of deoxyribose thereof. In addition, examples of the atom for, for example, ATP, CTP, GTP, and UTP include carbon atoms at the 2′- and 3′-positions of ribose thereof.

The nucleotide derivative which is capped with an electrochemically convertible structure to be used in the present invention can be produced using as a raw material a corresponding nucleotide or nucleoside.

In other words, base moieties such as purine and pyrimidine and sugar hydroxyl groups other than the atoms to which cappings are to be bound are selectively protected in an appropriate manner, and then the nucleotide derivative can be synthesized by addition of the removable group which is electrochemically removable or the substituent which is electrochemically substitutable.

The electrochemically convertible structures to be used for capping respective nucleoside 5′-triphosphates may be ones which can be used for distinguishing nucleotides having the respective structures from one another by the electrochemical conversion of the electrochemically convertible moiety and a structural change in the structure containing the moiety which is elicited by the electrochemical conversion.

For example, cappings corresponding to adenine (A), cytosine (C), guanine (G), and thymine (T) or uracil (U), respectively, may undergo electrochemical reduction or oxidation at different electric potentials.

For the substituent for determination held by the 4 kinds of the nucleotide derivatives which satisfy the above-mentioned condition, 4 different kinds of substituents for determination may be used. Alternatively, the same kind of any of them may be used, and this can be generally performed by using the same kind of substituents for determination each having a single kind of electrochemically convertible structure, while the position of atoms of the bases to which the substituent (for capping) is bound are different from one another depending on the kinds of the bases. Further, even if the positions of the atoms to which the substituent is introduced are the same, the same kind of electrochemically convertible structure can be used as long as the respective cappings corresponding to the kinds of the bases bound to ribose or deoxyribose undergo electrochemical reduction or oxidation at different electric potentials.

The electric potential at which the capping undergoes electrochemical reduction or oxidation is not limited as long as the value thereof is within the potential window of an electrode system which is defined by the kind of the electrode to be used and the solvent. The electric potential is generally −100 V to +100 V (vs. SCE), preferably −10 V to +10 V (vs. SCE), and more preferably about −1.2 V to +1.0 V (vs. SCE).

### (Analysis Method)

Next, a description will be made of procedures of the method of analyzing a nucleic acid base sequence of the present invention.

In advance of a first step of the method of analyzing a nucleic acid base sequence of the present invention, a complementary pair of a target nucleic acid and a primer is formed by hybridization.

The formation is achieved by mixing the target nucleic acid and the primer, destroying the secondary structures thereof by heat treatment, and cooling the mixture to a temperature lower than the melting temperature (Tm) of the primer.

Note that, in advance of the first step of the method of analyzing a nucleic acid base sequence of the present invention, a sample containing a promoter sequence for an RNA polymerase can be prepared by PCR amplification using a primer containing the promoter sequence, or by cloning a ligated product of the promoter sequence and a target nucleic acid using an appropriate host.

In addition, in advance of the first step of the method of analyzing a nucleic acid base sequence of the present invention, the following polymerase-immobilized electrode is prepared.

That is, in a case where the sample containing a target nucleic acid is a complementary pair of the target nucleic acid and a primer, a nucleic acid-dependent DNA polymerase-immobilized electrode is prepared by immobilizing a nucleic acid-dependent DNA polymerase onto an electroconductive substrate.

On the other hand, in a case where the sample containing a target nucleic acid contains a promoter sequence for an RNA polymerase, a nucleic acid-dependent RNA polymerase-immobilized electrode is prepared by immobilizing a nucleic acid-dependent RNA polymerase onto an electroconductive substrate.

For the electroconductive substrate, there can preferably be used materials which have high electroconductivity and have sufficient electrochemical stability under the condition where an electrode is used. Examples of such material constituting an electroconductive member may include metals, electroconductive polymers, metal oxides, and carbon materials.

In addition, the immobilization of a polymerase may be performed by any method known to those skilled in the art, which is used for physically capturing an enzyme in vicinity of an electroconductive substrate and which is used in preparation of an enzyme electrode. Specific examples of the method include the methods as described in the following items (1) to (3).

### (1) Covalent Bond Method

A functional group is directly introduced to a surface of an electroconductive substrate so that the functional group is bound to a polymerase via a covalent bond, to thereby immobilize the polymerase. Alternatively, an electroconductive substrate is brought into contact with a carrier so that the carrier is disposed thereon, and a functional group is introduced to the carrier so that the functional group and a polymerase is bound via a covalent bond, to thereby immobilize the enzyme.

Examples of the functional group which can be used for the covalent bond include hydroxyl group, carboxyl group, amino group, aldehyde group, hydrazino group, thiocyanate group, epoxy group, vinyl group, halogen, acid ester group, phosphate group, thiol group, disulfide group, dithiocarbamate group, dithiophosphate group, dithiophosphonate group, thioether group, thiosulfate group, and thiourea group.

Alternatively, the property of a thiol group of an alkyl thiol to act on and bind to a metal such as gold to easily form a monomolecular film (self-assembled monomolecular film) is utilized to bind an enzyme to a metal by a covalent bond via a functional group which has preliminarily introduced to an alkyl group of an alkyl thiol, to thereby immobilize the enzyme.

The covalent bond between the functional group which has preliminarily introduced to an alkyl group of alkyl thiol and the enzyme can be formed by, for example, using a bifunctional reagent.

Examples of a representative bifunctional reagent include glutaraldehyde, periodic acid, N,N′-o-phenylenedimaleimide, N-succinimidyl-4-(N-maleimidomethyl)cyclohexane-1-carboxylate, N-succinimidyl maleimide acetate, N-succinimidyl-4-maleimide butyrate, N-succinimidyl-6-maleimide hexanoate, N-sulfosuccinimidyl-4-maieimidomethylcyclohexane-1-carboxylic acid, N-sulfosuccinimidyl-3-maleimidobenzoic acid, N-(4-maleimidobutyryloxy)sulfosuccinimide sodium salt, N-(6-maleimidocaproyloxy)sulfosuccinimide sodium salt, N-(8-maleimidocapryloxy)sulfosuccinimide sodium salt, N-(11-maleimidoundecanoyloxy)sulfosuccinimide sodium salt, N[2-(1-piperazinyl)ethyl]maleimide dihydrochloride, and disulfone compounds such as a divinylsulfone compound.

In addition, examples of the carrier to be brought into contact with and disposed on an electroconductive substrate include agarose, an agarose decomposition product, κ-carageenan, agar, alginic acid, polyacrylamide, polyisopropyl acrylamide, polyvinyl alcohols, and copolymers thereof.

### (2) Adsorption Method (First Adsorption Method)

A polymerase is immobilized by a physical adsorption method utilizing a hydrophobic interaction or electrostatic interaction between an electroconductive substrate and the polymerase. In a case where the physical adsorption of a polymerase to an electroconductive substrate is impossible or insufficient, the polymerase can be immobilized thereto via a carrier to which the polymerase is physically adsorbed. Examples of such carrier which can be used include carriers composed of polyanion or polycation such as polyallylamine, polylysine, polyvinylpyridine, amino-modified dextrans such as DEAE-dextran, chitosan, polyglutamate, polystyrenesulfonic acid, and dextran sulfate. A polymerase is immobilized on a carrier by an ionic bond method utilizing the electrostatic interaction between the carrier and the polymerase, and the thus-obtained polymerase-immobilized carrier is brought into contact with an electroconductive substrate and disposed thereon.

### (3) Adsorption Method (Second Adsorption Method)

A polymerase is immobilized using various affinity tags which are used for facilitating purification of gene recombinant proteins. For example, a polymerase is immobilized using an epitope tag such as hemagglutinin (HA), FLAG, or Myc, GST, a maltose-binding protein, a biotinylated peptide, an oligohistidine tag, or the like.

The amount of the polymerase to be immobilized onto the polymerase-immobilized electrode of the present invention is not particularly limited, and can be widely changed.

Next, the thus-prepared polymerase-immobilized electrode is added with a sample containing a promoter sequence for an RNA polymerase or a complementary pair of a target nucleic acid and a primer. The sample containing a promoter sequence for an RNA polymerase or a complementary pair of a target nucleic acid and a primer is captured by the polymerase that exists on the polymerase-immobilized electrode.

In the first step of the method of analyzing a nucleic acid base sequence of the present invention, there is prepared a polymerase-immobilized electrode which is capturing a sample containing a complementary pair of a target nucleic acid and a primer or a sample containing a promoter sequence for an RNA polymerase. Then, the polymerase is allowed to coexist with the nucleotide derivatives according to the present invention.

The base moieties of the nucleotide derivatives are capped with structures having different electric signals obtained by electrochemical determination means, which correspond to adenine (A), cytosine (C), guanine (G), and thymine (T) (or uracil (U)), respectively. As a matter of course, multiple kinds of the nucleotide derivatives may be capped with the same structure as long as the respective nucleotide derivatives can be identified.

Examples of the nucleotide derivatives include nucleoside 5′-triphosphate derivatives, nucleoside 5′-diphosphate derivatives, nucleoside 5′-monophosphate derivatives, and nucleoside 3′-phosphate derivatives.

A mixture containing a polymerase and the nucleotide derivatives preferably contains various nucleoside 5′-triphosphate derivatives in equal concentrations.

As a consequence, in a case where a complementary pair of a target nucleic acid and a primer are used as a sample, a phosphate ester bond is formed between a hydroxyl group at the 3′-terminal of the primer in the complementary pair of the target nucleic acid and the primer (or elongated product thereof) and the 5′-phosphate group in the nucleoside 5′-triphosphate derivative containing bases complementary to the target nucleic acid.

At this time, pyrophosphoric acid is removed.

The elongation reaction by one base by the polymerase is known to terminate, in general, within 1 second, and especially in 1/500 second at the earliest in a case where DNA polymerase III derived from Escherichia coli is used. FIGS. 1A to 1C show an example of the above-mentioned processes.

On the other hand, in a case where the sample containing a promoter sequence for an RNA polymerase is used, transcription starts from the transcription initiation site which exists downstream of the promoter.

Next, in the second step of the method of analyzing a nucleic acid base sequence of the present invention, a polymerase-immobilized electrode is applied with voltage which gradually changes with time.

The voltage is changed in the negative direction in a case of a reduction reaction and in the positive direction in a case of an oxidation reaction with respect to the spontaneous potential. The voltage is changed in such a manner that: the voltage is swept at a constant rate from the spontaneous potential; or the voltage to be applied increases stepwise or like pulses.

When the voltage is changed such that the absolute value of electric potential always increases, the nucleotide derivatives undergo electrochemical conversion in the order of the absolute value of electric potential required therefor from the smallest to the largest depending on the kinds of the cappings which have been bound to the nucleotide derivatives.

An example of the reaction is shown in FIG. 1D. A reaction solution may be added with a supporting electrolyte of a kind which does not inhibit the activity of an enzyme and in a concentration which does not inhibit the activity of an enzyme upon the electrochemical conversion of the nucleotide derivatives. Examples of the supporting electrolyte include Na₂HPO₄, NaH₂PO₄, and KCl and Na₂HPO₄ and NaH₂PO₄ are preferably used because they also act as buffer solutions.

In the second step, an applied voltage value and a current value which flows through the electrode system are monitored. A reduction reaction or an oxidation reaction is elicited at the voltage depending on the kinds of the capping which has been bound to the nucleotide derivatives each containing a base complementary to that of the target nucleic acid, and electric current that accompanies the reaction can be observed.

A voltage which is applied to the electrode at the observation of the electric current that accompanies the reaction varies depending on the kind of the capping which has been bound to the nucleotide derivatives. Therefore, the value of the voltage can indicate the kind of the base at the 3′-terminal of an elongating strand, that is, the kind of the base in the target nucleic acid, which corresponds to the base.

In a case of analysis of a single base such as single nucleotide polymorphism, it can be performed by the first and second steps as mentioned above using a continuous base sequence in which a primer to be used is located adjacent to the site intended to be analyzed. Nucleoside 5′-triphosphate derivatives to be used in this case are not required to be a mixture of derivatives having base moieties of all of A, C, G, and T or U, respectively, and at least one kind of the derivative having the base constituting the polymorphism intended to be analyzed. When the single nucleotide polymorphism is analyzed in response thereto, the voltage to be applied to the polymerase-immobilized electrode is not necessarily be changed gradually with time, and a voltage required for the electrochemical conversion of the used nucleotide derivative may be applied.

In a case where a subsequent base of the target nucleic acid is intended to be detected, that is, in a case where a base sequence of the target nucleic acid is intended to be analyzed, the first and second steps as mentioned above may be repeated.

In this case, an operation of removing unreacted nucleoside 5′-triphosphate derivatives remaining in the solution is not necessarily performed between the first and second steps.

In addition, when a sufficient amount of the nucleoside 5′-triphosphate derivative is added to the system at the start, the nucleoside 5′-triphosphate derivative is not required to be supplemented even when the first step is repeated after the second step.

Note that when voltage is applied to electrochemically convert the cap structure modified by a nucleotide at the 3′-terminal of the elongating strand, there may be a case where unreacted nucleoside 5′-triphosphate derivatives remaining in the solution are also electrochemically converted on the electrode.

However, “contribution of error signal” generated by the electrochemical conversion of the unreacted nucleoside 5′-triphosphate derivative remaining in the solution on the electrode can be excluded or reduced, for example, as described below.

In general, dielectric conductivity inside a protein such as polymerase is different from that of water.

Therefore, there is a difference between the electric potential required for electrochemically converting the cap structure of a nucleotide derivative which has been added to the 3′-terminal of an elongating strand and the electric potential required for electrochemically converting the cap structure of an unreacted nucleoside 5′-triphosphate derivative remaining in the solution.

By distinguishing the difference as a difference in the applied voltage, the contribution of an error signal can be excluded.

In addition, the nucleotide derivative which has been added to the 3′-terminal of the elongating strand is captured in vicinity of the electroconductive substrate. In contrast, the unreacted nucleoside 5′-triphosphate derivative remaining in the solution floats in the solution.

Thus, there is a difference in diffusion coefficient between them. The difference in diffusion coefficient can be determined by, for example, the impedance method. Therefore, the difference in diffusion coefficient can be utilized for reducing the contribution of an error signal.

For example, by making the change with time of an applied voltage rapid, an electrochemical reaction of the bound nucleotide derivative which is captured in vicinity of the electroconductive substrate is performed before the unreacted nucleoside 5′ triphosphate derivative remaining in the solution is dispersed and reaches the electroconductive substrate.

Thus, the contribution of an error signal can be reduced.

Further, the phosphate group in the unreacted nucleoside 5′-triphosphate derivative remaining in the solution dissociates generally under a condition where a polymerase has a catalytic activity to have a negative charge. Therefore, when negative voltage is applied to the electrode to, for example, reduce a derivative at the terminal of an elongating strand, the unreacted nucleoside 5′-triphosphate derivative remaining in the solution undergoes electrostatic repulsion between the electrode so that it can not approach the electrode.

Thus, the detection by reducing the derivative at the terminal of an elongating strand is supposed to originally accompany with little contribution of an error signal.

In addition, even in a case of the detection by oxidizing the derivative at the terminal of an elongating strand, the unreacted nucleoside 5′-triphosphate derivative remaining in the solution can be kept away from the vicinity of the electrode by maintaining the electrode voltage negative for a certain time period before the application of oxidation electric potential, so the contribution of an error signal can be reduced.

Further, in a case of analyzing the single nucleotide polymorphism, that is, an aspect of the method of analyzing a nucleic acid base sequence of the present invention, it is effective to remove the unreacted nucleoside 5′-triphosphate derivative remaining in the solution by a washing operation of the electrode.

Next, a description will be made of the device for analyzing a nucleic acid base sequence of the present invention.

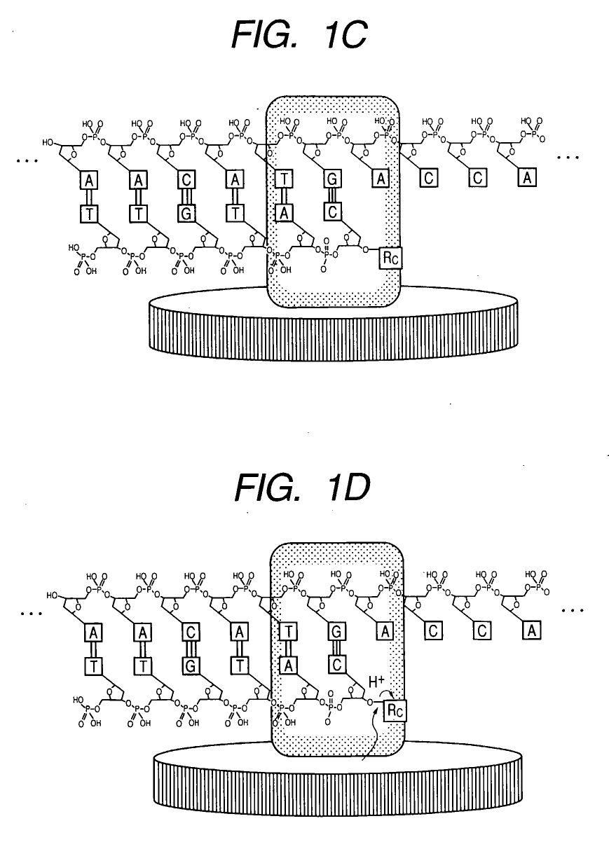

FIG. 2 shows an example of the information acquiring device of the present invention which is used for a DNA sequence analyzer.

The DNA sequence analyzer has an electrode system of a three-electrode cell composed of a polymerase-immobilized electrode (working electrode), a counter electrode, and a reference electrode which are connected to a potentiostat. The potentiostat is connected with a function generator for setting electrode voltage and a computer for determination and data processing. The voltage to be applied to the working electrode is programmed by the function generator and is applied to the working electrode through the potentiostat. The applied voltage and an electric current value observed at this time are sent to the computer in which they are collected. The computer identifies the kind of a nucleotide derivative which is bound to an elongating strand, that is, the kind of a base which is elongated, on the basis of the voltage applied to the polymerase-immobilized electrode when the electric current is observed.

### EXAMPLES

Hereinafter, the present invention will be described in more detail by way of examples. However, the methods of the present invention are not intended to be limited to these examples.

### Example 1

For target nucleic acids, NCBI Assay ID ss38346831 containing single base polymorphism <A/G> which is derived from human HLA gene was intended to be a model therefor, and single-stranded synthetic oligodeoxyribonucleotides represented by SEQ ID NOS: 1 and 2 were used. In addition, a synthetic oligodeoxyribonucleotide represented by SEQ ID NO: 3 was used as a primer.

A polymerase-immobilized electrode was prepared in such a manner that 50 μl of a buffer solution A of the following composition containing 2 units of T4 DNA polymerase (manufactured by Takara Holdings Inc.) was applied onto a glassy carbon electrode so that the polymerase was immobilized thereon by physical adsorption.

Buffer solution A

| | | |
|---|---|---|
| 33 | mM | Tris-acetate buffer solution |
| | | (pH 7.9) |
| 66 | mM | Potassium acetate |
| 10 | mM | Magnesium acetate |
| 0.5 | mM | Dithiothreitol |
| 0.01% | (w/v) | Bovine serum albumin |

The prepared polymerase-immobilized electrode was washed with the buffer solution A to remove unimmobilized polymerases.

First, 10 pmol of the target nucleic acids and 10 pmol of the primer were mixed in 50 μl of a TE buffer. The mixture was heated at 96° C. for 20 seconds and then allowed to cool at 25° C., to prepare complementary pairs of the primer and the target nucleic acids, respectively.

Next, the prepared polymerase-immobilized electrode was brought into contact with the mixture of the target nucleic acids and the primer, and the whole system was maintained at 37° C. for 5 minutes. After that, the polymerase-immobilized electrode was washed with the buffer solution A to remove the mixture of the target nucleic acids and the primer, which had not been captured by the polymerase-immobilized electrode.

An example of a nucleoside 5′-triphosphate derivative which is obtained by capping a nucleoside 5′-triphosphate having at least one base selected from adenine (A), cytosine (C), guanine (G), and thymine (T) with an electrochemically convertible structure is shown below.

Specifically, 2′-iodo-2′-deoxyadenosine-5′-triphosphate (2′I-dATP) (manufactured by JENA BIOSCIENCE GmbH) was used.

A 50 μM aqueous solution (pH 7.0) of 2′I-dATP was brought into contact with the polymerase-immobilized electrode, and the whole system was incubated at 37° C. for 10 seconds. Next, the polymerase-immobilized electrode was washed with a 33 mM Tris-acetate buffer solution (pH 7.9) to remove unreacted 2′I-dATP. Then, by using the polymerase-immobilized electrode as a working electrode, analysis was conducted using a DNA base sequence analyzer having a constitution as shown in FIG. 2. A platinum wire and a silver-silver chloride electrode were used as a counter electrode and a reference electrode, respectively. When a voltage to be applied was swept at a constant rate of 10 mV/sec from the spontaneous potential in the negative direction, a transient reduction current was observed only in the case where the synthetic DNA represented by SEQ ID NO: 1 was used as a target nucleic acid. No reduction current was observed in the case where the synthetic DNA represented by SEQ ID NO: 2 was used as a target nucleic acid. As a consequence, elongation of 2′I-dATP which is complementary to a target nucleic acid was able to be detected at the 3′-terminal of the primer in the complementary pair of the target nucleic acid and the primer only in the case where the target nucleic acid represented by SEQ ID NO: 1 was used. Further, it was found that the difference by single base in the target nucleic acids represented by SEQ ID NOS: 1 and 2 can be detected on the basis of the presence or absence of the reduction current that accompanies the electrochemical conversion.

### Example 2

For a target nucleic acid, a synthetic oligodeoxyribonucleotide represented by SEQ ID NO: 1 was used as a model. In addition, a synthetic oligodeoxyribonucleotide represented by SEQ ID NO: 4 was used as a primer. The polymerase-immobilized electrode prepared in Example 1 was used.

First, 10 pmol of the target nucleic acid and 10 pmol of the primer were mixed in 50 μl of a TE buffer. The mixture was heated at 96° C. for 20 seconds and then allowed to cool at 25° C. Then, by using the polymerase-immobilized electrode as a working electrode, analysis was conducted using a DNA base sequence analyzer having a constitution as shown in FIG. 2. A platinum wire and a silver-silver chloride electrode were used as a counter electrode and a reference electrode, respectively.

For a mixture of nucleoside 5′-triphosphate derivatives capped with different electrochemically convertible structures which correspond to adenine (A), cytosine (C), guanine (G), and thymine (T), respectively, the following materials were used.

2′-iodo-2′-deoxyadenosine-5′-triphosphate (2′I-dATP)

2-bromo-2′-deoxyguanosine-5′-triphosphate (2′Br-dGTP)

2′-chloro-2′-deoxythymidine-5′-triphosphate (2′Cl-dTTP)

2′-fluoro-2′-deoxycytidine-5′-triphosphate (2′F-dCTP)

A part of them is commercially available from JENA BIOSCIENCE GmbH, TriLink BioTechnologies, or the like.

In addition, they can also be synthesized according to the methods described in: Japanese Patent Application Laid-open No. H07-97391; Japanese Patent Publication No. H08-5908; Gruen M., et al., Nucleosides Nucleotides, 18, 137-151 (1999); “Oligonucleotide Synthesis; a practical approach”, M. J. Gait (ed), TRL PRESS (1984); and the like.

The mixture of the target nucleic acid and the primer was charged into the above-mentioned analyzer so that the mixture was brought into contact with the electrode system. The whole was maintained at 37° C. for 5 minutes, and the electrode system was washed with the buffer solution A to remove the mixture of the target nucleic acid and the primer, which had not been captured by the polymerase-immobilized electrode.

Next, an aqueous solution (pH 7.0) containing 50 μM each of 2′I-dATP, 2′Br-dGTP, 2′Cl-dTTP, and 2′F-dCTP was brought into contact with the polymerase-immobilized electrode, respectively, and maintained at 37° C.

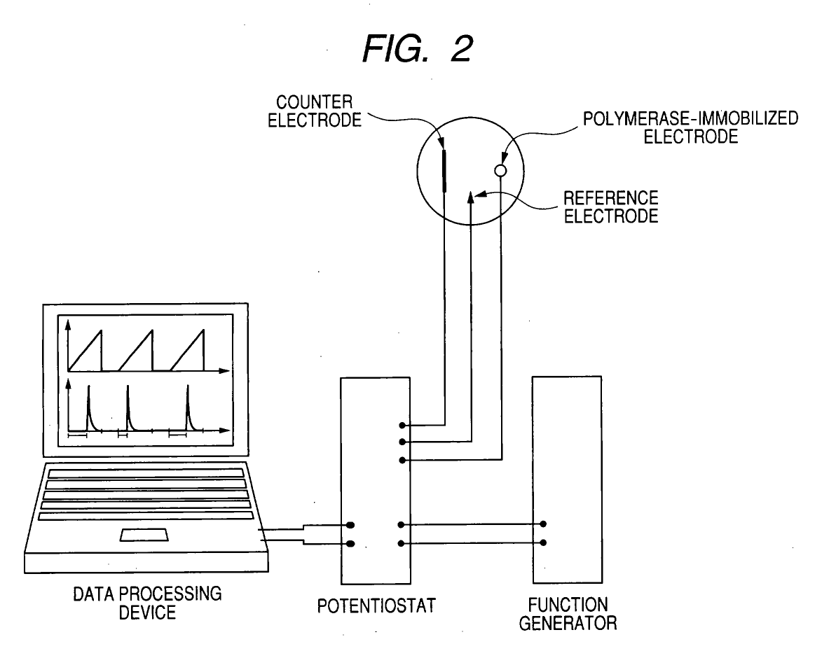

Then, a voltage which changed with time as shown in FIG. 3A was applied to the polymerase-immobilized electrode, and current values at this time were monitored. The change with time of the voltage as shown in FIG. 3A was repetition of changes composed of a phase in which the voltage value was swept at a constant rate from the spontaneous potential in the negative direction and a phase in which the voltage value is maintained at the spontaneous potential. FIG. 3A shows 5 repetition units from the start, and at this time, changes in current value as shown in FIG. 3B were observed.

A transient current value was observed for each repetition unit. However, the time periods from a start of sweeping the voltage to the observation of a peak current (τ1, τ2, τ3, τ4, and τ5) varied among respective repetition units, and the order thereof was found to be τ1>τ3=τ4>τ5>τ2.

The deoxynucleoside-phosphate derivatives, that is, 2′I-dAMP, 2′Br-dGMP, 2′Cl-dTMP, and 2′F-dCMP, were used for determination of the voltage required for the electrochemical conversion of the respective deoxynucleoside-phosphate derivatives. As a result, the order of magnitude of their absolute values was found to be 2′F-dCMP>2′Cl-dTMP>2′Br-dGMP>2′I-dAMP. From the results, the following was revealed.

(1) The signal obtained at τ1 that was a first repetition unit was derived from 2′F-dCMP having bound to the 3′-terminal.

(2) The signal obtained at τ2 that was a second repetition unit was derived from 2′I-dAMP having bound to the 3′-terminal.

(3) The signal obtained at τ3 that was a third repetition unit was derived from 2′Cl-dTMP having bound to the 3′-terminal.

(4) The signal obtained at τ4 that was a fourth repetition unit was derived from 2′Cl-dTMP having bound to the 3′-terminal.

(5) The signal obtained at τ5 that was a fifth repetition unit was derived from 2′Br-dGMP having bound to the 3′-terminal.

In other words, the base sequence which had been elongated from the primer was found to be 5′-CATTG . . . -3′. In addition, a corresponding base sequence of the target nucleic acid was found to be 3′-GTAAC . . . -5′. In each repetition unit, almost no influence due to liberated nucleoside 5′-triphosphate derivatives was observed, and the base sequence was found not to be elongated by a next base during the voltage application.

The results are supposed to be caused by the application of negative voltage, by which the liberated nucleoside 5′-triphosphate derivatives each having a negative charge can not approach the electrode so that no electrode reaction occurs, and, also, can not approach the DNA polymerase so that they do not serve as the substrates for the elongation reaction.

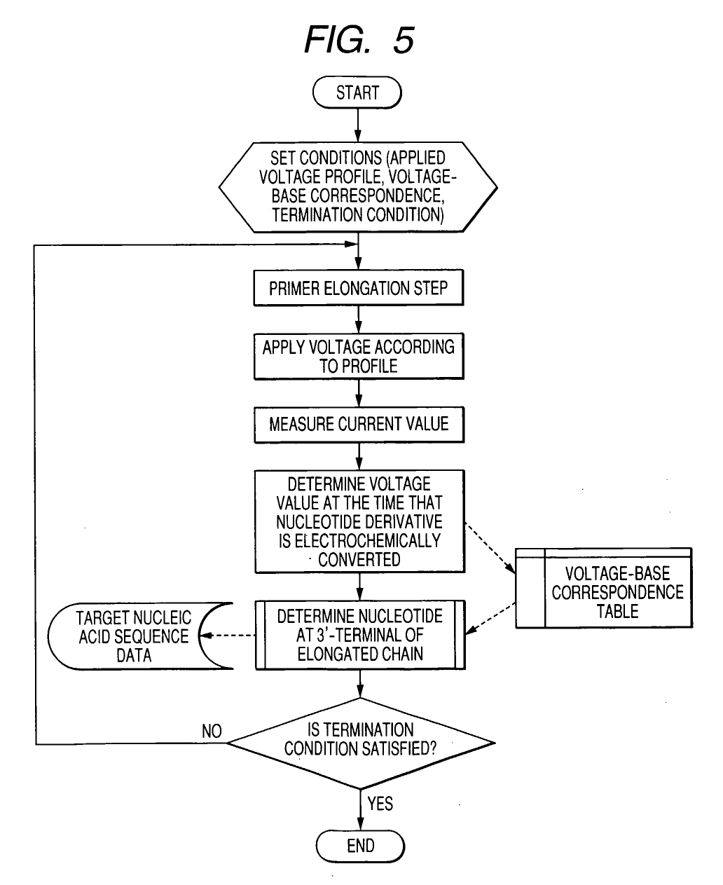

Note that the determination according to the signal may be performed by a program which has preliminarily installed in a computer to store the analysis results in a memory or to display or print out the analysis results. In addition, the application of the voltage upon measurement is not limited to the manner shown in FIGS. 3A and 3B, and there can be used an application method in which the voltage is increased stepwise, or a method of successively applying pulses of a certain voltage. FIG. 5 shows an exemplary flow chart showing a flow of the program. First, conditions are set. The conditions to be set herein include a profile of an applied voltage, termination conditions, and the like. Then, a primer elongation step is performed while, for example, the voltage is maintained at the spontaneous potential. After that, voltage is applied under the set condition and a current value is measured at the same Lime. Then, the voltage value at which a nucleotide derivative is electrochemically converted is determined. Then, the voltage value was compared with information stocked in a database (for example, in the case of A, values such as a current value at removal) to identify the nucleotide at the 3′-terminal of the elongated strand. The identified information is successively stocked. When the termination condition is satisfied, the determination is terminated.

### Example 3

For a nucleotide derivative having an electrochemically convertible moiety, the following was used in the present invention.

That is, a nucleotide derivative, 2′-deoxy-3′-(2-azidomethyl)benzoyl-nucleoside 5′-triphosphate, represented by the following formula was used.

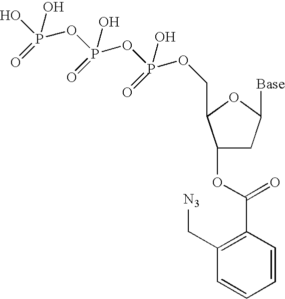

Provided that, in the formula, the term “Base” represents a base which generally constitutes a nucleic acid, such as adenine, guanine, cytosine, uracil, and thymine.

The nucleotide derivative can be synthesized according to the information disclosed in Tetrahedron Letters, 42 (2001), 1069-1072 and Acta Biochimica et Biophysica Academiae Scientiarum Hungaricae, 16 (1981), 131-133 in a manner, for example, as shown below.

That is, first, methyl 2-methylbenzoate (1 equivalent) was allowed to react with bromosuccinimide (1.1 equivalent) in a tetrachloromethane solvent for 1 hour while the mixture was refluxed. In this case, benzoyl peroxide (0.02 equivalent) was used as a catalyst.

From the reaction, methyl 2-(bromomethyl)benzoate was produced.

Next, methyl 2-(bromomethyl)benzoate (1 equivalent) and tetramethylguanidinium azide (1.5 equivalent) were refluxed in a carbon tetrachloride-methanol solvent mixture (1:1, v/v) for 1.5 hours to produce methyl 2-(azidomethyl)benzoate.

Next, the methyl 2-(azidomethyl)benzoate (1 equivalent) was slowly stirred in a solvent mixture (1:1, v/v) of a 2 M sodium hydroxide aqueous solution and methanol at room temperature for 30 minutes, to thereby produce 2-(azidomethyl)benzoic acid.

Next, the 2-(azidomethyl)benzoic acid (1 equivalent) was refluxed in thionyl chloride (1.5 equivalent) for 1 hour to produce 2-(azidomethyl)benzoyl chloride.

The 2-(azidomethyl)benzoyl chloride and nucleosides in which active sites such as bases, 5′-hydroxyl groups, or the like are protected were slowly stirred for 2 hours in a pyridine solvent. By this, a 2-(azidomethyl)benzoyl group was introduced into the hydroxyl group at the 3′-position of a D-ribose which constitutes the nucleoside, to thereby produce a nucleoside derivative.

Next, protective groups of the hydroxyl groups at 5′-position of the nucleoside derivative were removed. After that, 1 equivalent of the resultant product was mixed with phosphoryl chloride (1.5 equivalent) in a dehydrated phosphoric acid, trimethyl ester solvent, and the whole was stirred at 0° C. for 1.5 hours.

Next, the mixture was added with a mixture solution (10:1, v/v) of a dehydrated dimethylformamide (5 M) solution of bis-tri-n-butylammonium pyrophosphate and tributylamine, and the mixture was stirred for 1 minute. After that, the mixture was added with 1 M triethylamine-carbonate buffer solution (pH 7.5).

After evaporation, the mixture was subjected to fractionation using anionic chromatography to fractionate a target fraction. The target fraction was subjected to deprotection, to thereby obtain the target nucleotide derivative.

The nucleotide derivative represented by the above-mentioned formula has a triphosphate being bound through an ester bond at the 5′-position of D-ribose, so it serves as a substrate for polymerase and thus is added to the 3′-OH terminal of an elongating strand.

The nucleotide derivative added to the 3′-OH terminal of an elongated strand has a (2-azidomethyl)benzoyl group being added to the 3′-position of D-ribose, so it can inhibit further nucleotide addition by polymerase.

The electrochemically convertible moiety in the nucleotide derivative represented by the above mentioned formula is an azide (N₃—) group.

Bioelectrochemistry and Bioenergetics 26 (1991) 441-455 discloses a finding that multiple kinds of nucleosides each having an azide group have different values of oxidation-reduction electric potential of the azide groups.

From the finding, the inventors of the present invention assume that the nucleotide derivatives of the present example similarly have different values of oxidation-reduction electric potential of the azide groups thereof depending on the kinds of bases thereof.

If required, the (2-azidomethyl)benzoyl group moieties may be further modified with different functional groups depending on the kinds of the base so that the value of the oxidation-reduction electric potential significantly may differ from one another depending on the kinds of the base.

Bioelectrochemistry and Bioenergetics 26 (1991) 441-455 discloses a finding that an azide group is converted into an amino group by electrochemical reduction.

In addition, Tetrahedron Letters 42 (2001) 1069-1072 discloses an example in which a (2-azidomethyl)benzoyl group is used as a protective group for a sugar hydroxyl group. It also discloses a finding that this protective group undergoes a removal reaction to regenerate the sugar hydroxyl group when the azide group is converted to an amino group by addition of a chemically reducing agent.

Thus, when the (2-azidomethyl)benzoyl group being added to the 3′-position of D-ribose in the nucleotide derivative of the present example is electrochemically reduced, an azide group thereof is converted to an amino group and then undergoes a removal reaction, to thereby regenerate the hydroxyl group at the 3′-position of the D-ribose.

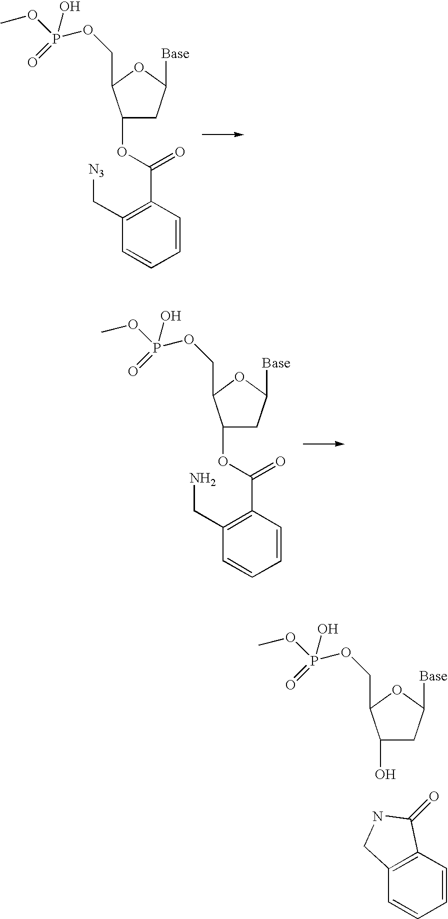

When the hydroxyl group is regenerated at the 3′-position of an elongated strand by the removal reaction elicited after the electrochemical reduction, it becomes possible to add a new nucleotide by the catalyst action of the polymerase.

Thus, the nucleotide derivative of the present example has an electrochemically convertible moiety, so it can preferably be used in the present invention.

### Example 4

As an example of a nucleotide derivative having an electrochemically convertible moiety, a nucleotide derivative represented by the following formula, that is, 2′-O-(4-methoxy-2,2,6,6-tetramethyl-1-piperidinyl)-nucleoside-5′-triphosphate is shown below.

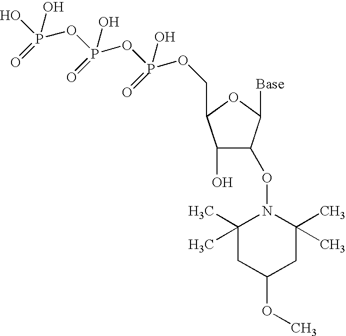

Nucleosides, Nucleotides & Nucleic Acids (2004), 23 (11), 1723-1738 describes a synthesis example of 2′-O-(4-methoxyl-2,2,6,6-tetramethyl-1-piperidinyl)-5-methyl-uridine. By referring to the document, 2,2,6,6-tetramethyl-1-piperidinyloxy (TEMPO) groups can each be introduced into the 2′-position of D-ribose of various nucleosides.

Further, according to the method disclosed in Acta Biochimica et Biophysica Academiae Scientiarum Hungaricae 16 (1981) 131-133, a triphosphate can be allowed to bind to the 5′-position of D-ribose via an ester bond, to thereby obtain a target nucleotide derivative.

The nucleotide derivative represented by the above-mentioned formula has a triphosphate being bound to the 5′ position of D-ribose thereof via an ester bond, so it serves as a substrate for polymerase and is added to the 3′-OH terminal of an elongated strand.

The nucleotide derivative added to the 3′-OH terminal of an elongated strand has a methoxy-2,2,6,6-tetramethyl-1-piperidinyloxy (methoxy-TEMPO) group being added to the 2′-position of D-ribose thereof. Therefore, the nucleotide derivative can inhibit further nucleotide addition by polymerase owing to steric hindrance thereof.

The electrochemically convertible moiety in the nucleotide derivative represented by the above-mentioned formula is a methoxy-TEMPO group. TEMPO is known to be a stable radial species, and undergo a removal reaction by electrochemical reduction to regenerate the hydrogen at the 2′-position of D-ribose.

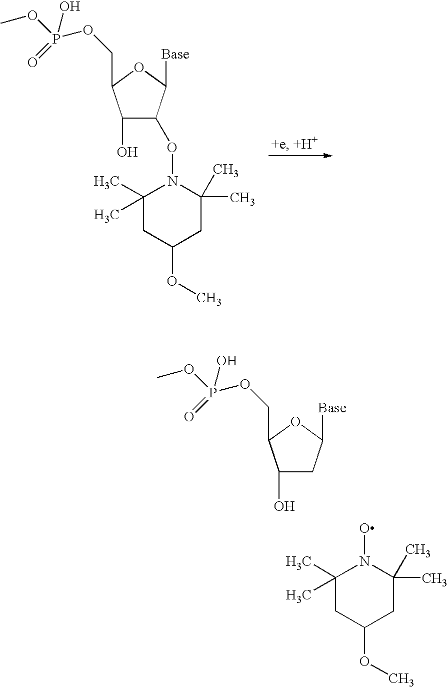

By this, it becomes possible to add a new nucleotide to the hydroxyl group at the 3′-position of an elongated strand by the catalyst action of the polymerase.

Thus, the nucleotide derivative of the present example has an electrochemically convertible moiety, so it can preferably be used in the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims priority from Japanese Patent Application No. 2005-380326 filed on Dec. 28, 2005, which is hereby incorporated by reference herein.

1. A method of acquiring information regarding a base sequence, comprising:
preparing multiple kinds of nucleotide derivatives each having a removable moiety which is electrochemically removable from the nucleotide derivatives, a double-stranded sample made of a target nucleic acid and a primer, and a polymerase;
elongating the primer by one base with one kind of the nucleotide derivatives by allowing the sample, the polymerase, and the nucleotide derivatives to coexist in a solvent;
applying a voltage to the sample; and
detecting an electric signal due to removal of the removable moiety from the nucleotide derivative which has been incorporated in the primer,
wherein the nucleotide derivative is capped with the removable moiety such that it acts as a chain terminator on its incorporation into a growing nucleic acid strand.

2. A method of acquiring information regarding a base sequence, comprising:
preparing a sample comprising a target nucleic acid hybridized with a primer or a sample comprising a target nucleic acid containing a promoter sequence, a polymerase, and a nucleotide derivative having an electrochemically convertible moiety capable of undergoing a reaction of removal from the nucleotide derivative, substitution in the nucleotide derivative, or addition to the nucleotide derivative owing to the electrochemical conversion;
allowing the sample, the polymerase, and the nucleotide derivative to coexist in a solvent; and
detecting whether the nucleotide derivative is introduced into the primer or a transcription product of the target nucleic acid or not by electrochemically converting the electrochemically convertible moiety,
wherein the nucleotide derivative is capped with the electrochemically convertible moiety such that it acts as a chain terminator on its incorporation into a growing nucleic acid strand.

3. A method of acquiring information regarding a base sequence, comprising:
preparing a sample comprising a target nucleic acid hybridized with a primer or a sample comprising a target nucleic acid containing a promoter sequence, a polymerase, and multiple kinds of nucleotide derivatives each of which has an electrochemically convertible moiety capable of undergoing a reaction of removal from the nucleotide derivative, substitution in the nucleotide derivative, or addition to the nucleotide derivative owing to the electrochemical conversion and which are electrochemically distinguishable from one another;
allowing the sample, the polymerase, and the multiple kinds of the nucleotide derivatives to coexist in a solvent; and
identifying, among the multiple kinds of the nucleotide derivatives, a nucleotide derivative which has been introduced into the primer or a transcription product of the target nucleic acid by electrochemically converting the electrochemically convertible moiety thereof,
wherein each of the nucleotide derivatives is capped with the electrochemically convertible moiety such that it acts as a chain terminator on its incorporation into a growing nucleic acid strand.

4.-7. (canceled)

8. A base sequence information acquiring method for acquiring information regarding a base at an information acquiring position in a target nucleic acid, comprising the steps of:
(1) preparing: the target nucleic acid; a primer which recognizes at least a part of a 3′-side region including a base prior in a 3′-side direction to a base at an information acquiring position in the target nucleic acid, and which hybridizes with the 3′-side region; a polymerase; and a nucleotide derivative having an electrochemically convertible moiety capable of undergoing a reaction of removal from the nucleotide derivative, substitution in the nucleotide derivative, or addition to the nucleotide derivative owing to the electrochemical conversion;
(2) hybridizing the 3′-side region including the base adjacent in a 3′-direction to the base at the information acquiring position in the target nucleic acid, with the primer;
(3) allowing the target nucleic acid having the hybridized region to coexist with the nucleotide derivative under a presence of the polymerase; and
(4) acquiring information regarding the base at the information acquiring position by detecting occurrence or absence of incorporation of the nucleotide derivative into a 3′-terminal position of a strand including the primer, which position corresponds to the information acquiring position, based on electrochemical conversion of the electrochemically convertible moiety of the nucleotide derivative,
wherein the nucleotide derivative is capped with the electrochemically convertible moiety such that it acts as a chain terminator on its incorporation into a growing nucleic acid strand.

9. The base sequence information acquiring method according to claim 8, wherein the hybridization is performed using a primer which recognizes the entire 3′-side region.

10. The base sequence information acquiring method according to claim 8, wherein the primer recognizes and hybridizes with a part of the 3′-side region, and the entire 3′-side region is made double-stranded by elongating a 3′-terminal of the primer hybridized with the nucleic acid.

11. The base sequence information acquiring method according to claim 8, wherein:
the target nucleic acid is a DNA;
the polymerase to be used is a DNA polymerase;
the nucleotide derivative to be used comprises at least one of a 2′-deoxyadenosine 5′-triphosphate derivative, a 2′-deoxycytidine 5′-triphosphate derivative, a 2′-deoxyguanosine 5′-triphosphate derivative, and a 2′-deoxythymidine 5′-triphosphate derivative each of which has an electrochemically convertible moiety and which are electrochemically distinguishable from one another.

12. A base sequence analyzing method for analyzing a base sequence of a subject region to be analyzed in a target nucleic acid, comprising the steps of:
(1) preparing: the target nucleic acid; a primer which recognizes a 3′-side region including a base adjacent in a 3′-side direction to the subject region to be analyzed in the target nucleic acid, and which hybridizes with the 3′-side region; a DNA polymerase; and a set of nucleotide derivatives including a 2′-deoxyadenosine 5′-triphosphate derivative, a 2′-deoxycytidine 5′-triphosphate derivative, a 2′-deoxyguanosine 5′-triphosphate derivative, and a 2′-deoxythymidine 5′-triphosphate derivative each of which has an electrochemically convertible moiety capable of undergoing a reaction of removal from the nucleotide derivative, substitution in the nucleotide derivative, or addition to the nucleotide derivative owing to the electrochemical conversion and which are electrochemically distinguishable from one another;
(2) hybridizing the target nucleic acid with the primer;
(3) elongating the primer by one base by allowing the target nucleic acid hybridized with the primer, the set of nucleotide derivatives, and the DNA polymerase to coexist so that a nucleotide derivative selected from the set of nucleotide derivatives is incorporated into a 3′-terminal of the primer hybridized with the target nucleic acid;
(4) identifying a base in the subject region to be analyzed which is present at a position corresponding to the nucleotide derivative which has been incorporated into the 3′-terminal of the elongated primer, by identifying the incorporated nucleotide derivative based on electrochemical conversion of the electrochemically convertible moiety in the incorporated nucleotide derivative; and
(5) repeating the steps (3) and (4) depending on the number of bases when the number of bases in the subject region to be analyzed is 2 or more, wherein
an additional elongation reaction of the elongated primer by the DNA polymerase is inhibited after the primer is elongated by one base in the step (3) owing to a presence of the electrochemically convertible moiety, and an additional elongation reaction of the elongated primer by the DNA polymerase is allowed to proceed after the electrochemical conversion of the electrochemically convertible moiety in the step (4) owing to a reaction of removal from, substitution in, or addition to the incorporated nucleotide derivative,
wherein each of the nucleotide derivatives is capped with the electrochemically convertible moiety such that it acts as a chain terminator on its incorporation into a growing nucleic acid strand.

13. A base sequence analyzing method for analyzing a base sequence of a subject region to be analyzed in a target nucleic acid, comprising the steps of:
(1) preparing: an RNA polymerase; the target nucleic acid containing a promoter sequence for the RNA polymerase; and a set of nucleotide derivatives including an adenosine 5′-triphosphate derivative, a cytidine 5′-triphosphate derivative, a guanosine 5′-triphosphate derivative, and a uridine 5′-triphosphate derivative each of which has an electrochemically convertible moiety capable of undergoing a reaction of removal from the nucleotide derivative, substitution in the nucleotide derivative, or addition to the nucleotide derivative owing to the electrochemical conversion and which are electrochemically distinguishable from one another;
(2) transcribing by one base the target nucleic acid which is located downstream of the promoter sequence by allowing the target nucleic acid, the set of nucleotide derivatives, and the RNA polymerase to coexist so that a nucleotide derivative selected from the set of nucleotide derivatives is incorporated into the target nucleic acid;
(3) identifying a base in the target nucleic acid, which is present at a position corresponding to the nucleotide derivative which has been incorporated into a 3′-terminal of the transcription product, by identifying the incorporated nucleotide derivative based on electrochemical conversion of the electrochemically convertible moiety in the incorporated nucleotide derivative;
(4) repeating the steps (2) and (3) depending on the number of bases when the number of bases in the subject region to be analyzed is 2 or more, wherein
an additional transcription reaction by the RNA polymerase is inhibited after the target nucleic acid is transcribed by one base in the step (2) owing to a presence of the electrochemically convertible moiety, and an additional transcription reaction by the RNA polymerase is allowed to proceed after the electrochemical conversion of the electrochemically convertible moiety in the step (3) owing to a reaction of removal from, substitution in, or addition to the incorporated nucleotide derivative,
wherein each of the nucleotide derivatives is capped with the electrochemically convertible moiety such that it acts as a chain terminator on its incorporation into a growing nucleic acid strand.

14. The base sequence analyzing method according to claim 12, wherein the polymerase is immobilized onto an electrode, and the electric signal is determined using the electrode.

15. (canceled)

16. The method of acquiring information regarding a base sequence according to claim 2, wherein the allowing step and the detecting step are repeated successively using the same electrode.

17. The method of acquiring information regarding a base sequence according to claim 2, wherein the electrochemically convertible moiety is converted by applying a voltage to a working electrode as swept in a negative direction.

18. The method of acquiring information regarding a base sequence according to claim 1, wherein the removable moiety is removed from the nucleotide derivative as a result of an electrochemical reaction caused by applying the voltage.

19. The base sequence analyzing method according to claim 13, wherein the polymerase is immobilized onto an electrode, and the electric signal is determined using the electrode.


SEQUENCE LISTING (4 sequences)

SEQ ID NO: 1 - DNA, 120 bp, Artificial
  Target DNA model #1
aggattataaatcatgctgctataaagacacatgcacacgcatgtttattacagcactattcacgatagcaaagacttggaaccaacccaaatgtccaacaatgatagactggattaaga

SEQ ID NO: 2 - DNA, 120 bp, Artificial
  Target DNA model #2
aggattataaatcatgctgctataaagacacatgcacacgcatgtttattgcagcactattcacgatagcaaagacttggaaccaacccaaatgtccaacaatgatagactggattaaga

SEQ ID NO: 3 - DNA, 30 bp, Artificial
  Primer
tccaagtctttgctatcgtgaatagtgctg

SEQ ID NO: 4 - DNA, 25 bp, Artificial
  primer
gccacattttcttaatccagtctat